(19) United States
(12) Patent Application Publication
(10) Pub. No.: US 2021/0331136 A1
(43) Pub. Date: Oct. 28, 2021

(54) MONOLITHIC CATALYSTS FOR EPOXIDATION

(71) Applicant: BASF CORPORATION, Florham Park, NJ (US)

(72) Inventors: Shiang SUNG, Iselin, NJ (US); Steven GAIK, Southfield, MI (US); Marco BOSCH, Ludwigshafen (DE); Andrey KARPOV, Ludwigshafen (DE)

(73) Assignee: BASF CORPORATION, Florham Park, NJ (US)

(21) Appl. No.: 16/479,840

(22) PCT Filed: Jan. 22, 2018
(86) PCT No.: PCT/US2018/014669

§ 371 Date: Jul. 22, 2019

Related U.S. Application Data
(60) Provisional application No. 62/449,908, filed on Jan. 24, 2017.

(51) Int. Cl. B01J 19/24; B01J 23/50; B01J 23/78; B01J 35/04; B01J 37/02; B01J 8/04; C07D 301/03

(52) U.S. Cl. CPC B01J 19/2485; B01J 23/50; B01J 23/78; B01J 35/04; B01J 2219/2438; B01J 37/0236; B01J 8/0492; C07D 301/03; B01J 2219/2428; B01J 37/0215

(57) ABSTRACT

A catalyst bed contains one or more segments of monolithic catalyst, wherein the monolithic catalyst includes a mono-lithic honeycomb structure and a layer of catalyst coating the honeycomb structure; the honeycomb structure contains a plurality of channels aligned side by side; and each channel includes an inlet positioned at a first terminus of the channel, an outlet positioned at a second terminus of the channel, and openings positioned along the channel in the direction of fluid flow through the channel for transverse fluid flow in and/or out of the channel.

### CROSS-REFERENCE TO RELATED APPLICATIONS

This application claims the benefit of priority to U.S. Provisional Application No. 62/449,908, filed on Jan. 24, 2017, the contents of which are incorporated herein by reference in their entirety.

### FIELD

The present technology is generally related to the field of monolithic catalysts. More particularly, the technology relates to monolithic catalysts for direct epoxidation.

### SUMMARY

In one aspect, provided herein are catalyst beds containing one or more segments of monolithic catalyst containing:
a monolithic honeycomb structure including a plurality of channels aligned side by side wherein each channel includes an inlet positioned at a first terminus of the channel, an outlet positioned at a second terminus of the channel, and openings positioned along the channel in the direction of fluid flow through the channel for transverse fluid flow in and/or out of the channel; and
a layer of catalyst coating the honeycomb structure.

In some embodiments, each of the openings is accompanied by a projection of channel wall toward the interior of the channel. In some embodiments, fluid flow is turbulent through each channel. In some embodiments, accumulation of heat is minimized or avoided within the channels. In some embodiments, the layer of catalyst coats the interior of each channel. In some embodiments, the layer of catalyst contains a refractory metal oxide support impregnated with metal. In some embodiments, the refractory metal oxide support contains a compound selected from alumina, silica, zirconia, titania, or a combination of any two or more thereof. In some embodiments, the layer of catalyst contains about 1 wt. % to about 50 wt. % metal. In some embodiments, the layer of catalyst contains about 10 wt. % to about 30 wt. % metal. In some embodiments, the layer of catalyst contains an alumina-based support impregnated with a metal selected from silver, copper, cobalt, nickel, or gold, or a combination of any two or more thereof. In some embodiments, the monolithic honeycomb structure contains cordierite, steel, or aluminum. In some embodiments, each of the one or more segments is about 7 centimeters to about 20 meters in length. In some embodiments, each of the one or more segments is about 15 centimeters to about 25 centimeters in length. In some embodiments, the catalyst bed is partitioned into one or more zones. In some embodiments, the catalyst bed further contains catalyst pellets. In some embodiments, the catalyst pellets and the one or more segments of monolithic catalyst are located in separate zones. In some embodiments, a zone containing one or more segments of monolithic catalyst is positioned to encounter fluid flow before a zone containing catalyst pellets. In some embodiments, the catalyst pellets and the one or more segments of monolithic catalyst are located in separate zones in an alternating pattern. In some embodiments, the catalyst bed contains two or more segments of monolithic catalyst, and the catalyst bed further contains a gap devoid of catalyst positioned between each of the two or more segments of monolithic catalyst.

In another aspect, provided herein are catalyst beds for the preparation of ethylene oxide.

In another aspect, provided herein are monolithic catalysts containing:
a monolithic honeycomb structure including a plurality of channels aligned side by side; and each channel includes an inlet positioned at a first terminus of the channel, an outlet positioned at a second terminus of the channel, and openings positioned along the channel in the direction of fluid flow through the channel for transverse fluid flow in and/or out of the channel; and
a layer of catalyst coating the honeycomb structure.

In some embodiments, each of the openings is accompanied by a projection of channel wall toward the interior of the channel. In some embodiments, fluid flow is turbulent through each channel. In some embodiments, accumulation of heat is minimized or avoided within the channels. In some embodiments, the layer of catalyst coats the interior of each channel. In some embodiments, the layer of catalyst contains a refractory metal oxide support impregnated with metal. In some embodiments, the refractory metal oxide support contains a compound selected from alumina, silica, zirconia, titania, or a combination of any two or more thereof. In some embodiments, the layer of catalyst contains about 1 wt. % to about 50 wt. % metal. In some embodiments, the layer of catalyst contains about 10 wt. % to about 30 wt. % metal. In some embodiments, the layer of catalyst contains an alumina-based support impregnated with a metal selected from silver, copper, cobalt, nickel or gold, or a combination of two or more thereof. In some embodiments, the monolithic honeycomb structure contains cordierite, steel, or aluminum.

In another aspect, provided herein are methods to prepare ethylene oxide, the method including:
contacting a feed gas containing ethylene with a monolithic catalyst to form ethylene oxide;
wherein:
the monolithic catalyst includes:
a monolithic honeycomb structure including a plurality of channels, each channel including openings positioned along the channel in the direction of fluid flow through the channel; and
a layer of catalyst coating the monolithic honeycomb structure.

In some embodiments, each of the openings is accompanied by a projection of channel wall toward the interior of the channel. In some embodiments, fluid flow is turbulent through each channel. In some embodiments, accumulation of heat is minimized or avoided within the channels. In some embodiments, the layer of catalyst coats the interior of each channel. In some embodiments, the layer of catalyst contains a refractory metal oxide support impregnated with metal. In some embodiments, the refractory metal oxide support contains a compound selected from alumina, silica, zirconia, titania, or a combination of any two or more thereof. In some embodiments, the layer of catalyst contains about 1 wt. % to about 50 wt. % metal. In some embodiments, the layer of catalyst contains about 10 wt. % to about 30 wt. % metal. In some embodiments, the layer of catalyst contains an alumina-based support impregnated with a metal selected from silver, copper, cobalt, nickel or gold, or a combination of two or more thereof. In some embodiments, the monolithic honeycomb structure contains cordierite, steel, or aluminum.

In another aspect, provided herein are methods to prepare a monolithic catalyst, the method including:
coating a monolithic honeycomb structure with a slurry of supported catalyst to form a coated honeycomb structure; and
drying the coated honeycomb structure with heated forced air to produce the monolithic catalyst;
wherein:
the monolithic honeycomb structure includes a plurality of channels, each channel including openings positioned along the channel in the direction of fluid flow through the channel; and
the supported catalyst contains a refractory metal oxide support impregnated with metal.

In some embodiments, the coating is performed by dipping the monolithic honeycomb structure into the slurry of supported catalyst. In some embodiments, the coating is performed by applying a wash-coat of supported catalyst to the monolithic honeycomb structure. In some embodiments, the coating step forms a layer of supported catalyst on the interior of each channel.

In another aspect, provided herein are methods to prepare a monolithic catalyst, the method including:
coating a monolithic honeycomb structure with a layer of alumina-based support to form a pre-coated monolithic honeycomb structure;
impregnating the layer of alumina-based support with a metal catalyst to form an impregnated monolithic honeycomb structure; and
drying the impregnated monolithic honeycomb structure with heated forced air to produce the monolithic catalyst;
wherein the monolithic honeycomb structure includes a plurality of channels, each channel including openings positioned along the channel in the direction of fluid flow through the channel.

In some embodiments, the coating step and impregnating step form a layer of alumina-based support impregnated with metal catalyst on the interior of each channel.

### BRIEF DESCRIPTION OF THE DRAWINGS

FIG. 1 depicts a pressure v. flow rate comparison of packed beds (solid lines) of specified packing height, and monoliths (dashed lines) of comparable bed height at identical flow conditions. Data shown in parentheses as “XXX/Y” refers to channels per square inch (XXX) and channel wall thickness in mils (Y).

FIG. 2 depicts cross-sectional side views of non-limiting sample loading arrangements for reactor tube loading arrangements. Monolith samples (left) and powder bed samples (right) were each loaded on top of a corundum powder guard bed which set the overall pressure drop for each reactor tube.

FIGS. 3A, 3B, 3C, and 3D are charts comparing monolith samples with production catalyst powder beds in the absence of combustion moderator. Monoliths and powders are operated at their optimum temperatures to allow comparison of the best measured performances for each geometry. The charts represent measured ethylene oxide selectivity vs. gas hourly space velocity (GHSV) (FIG. 3A), measured turnover frequency (TOF) vs. GHSV (FIG. 3B), and conversion vs. GHSV (FIG. 3D) for the samples listed in the table (FIG. 3C). Data point labels refer to the sample number. GHSV was varied by changing feed flow rate. Lines connect data points of identical bed height.

FIG. 4 is an illustration of various monolith channel structures for the (A) SC geometry, (B) LS geometry, and (C) LS/PE combination geometry.

FIGS. 5A, 5B, and 5C are various illustrations of geometry A₁ for a packed pellet bed. FIG. 5A depicts a “hot spot” in the center of a reactor tube with cylindrical pellets arranged side by side touching at their tangent surfaces as shown in B. The shape and dimensions of the thermal contact area for inter-pellet heat transfer is shown in C.

FIGS. 6A, 6B, and 6C are various illustrations of geometry A₂ for packed pellet bed. FIG. 6A depicts a “hot spot” in the center of a reactor tube with cylindrical pellets arranged end to end touching at their faces, the thermal contact areas of which are shown in FIG. 6B. The corresponding shape and dimensions of the thermal contact area for inter-pellet heat transfer is shown in FIG. 6C.

FIGS. 7A, 7B, and 7C are various illustrations for the geometry for a metallic monolith bed. FIG. 7A depicts a “hot spot” in the center of a reactor tube containing a single monolith core with channels parallel to the reactor tube axis and gas flow direction. A top view of the monolith channels is shown in FIG. 7B. FIG. 7C depicts the channel walls of B viewed along the direction of the heat flux vector. The heat transfer areas A₁ and A₂ from the packed bed geometries are overlaid to illustrate amount of material (i.e. the edges of the foil walls) available for heat transfer in a monolith.

FIG. 8 depicts a non-limiting example of a hybrid reactor geometry in which a series of monolith segments are placed atop a conventional pellet bed within a reactor tube.

FIG. 9 depicts a comparison of turnover frequency for monolith, powder, and hybrid bed reactor geometries as a function of gas hourly space velocity at 200° C. in the absence of moderator.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Provided herein are catalyst beds that avoid the use of dedicated heat exchangers. The catalyst beds described herein have superior mass and heat transport within the catalyst beds compared to packed catalyst beds consisting essentially of catalyst pellets or other media. This superior mass and heat transport results in lower temperatures experienced within the catalyst beds described herein. Such lower temperatures may result in longer lifetime and higher selectivity for the catalyst within the catalyst beds described herein compared to conventional packed catalyst beds. Lower catalyst loading may be employed to achieve comparable or higher yields of desired reaction product compared to conventional packed catalyst beds.

The catalyst bed may contain one or more segments of monolithic catalyst. In some embodiments, the catalyst bed consists essentially of one or more segments of monolithic catalyst. In some embodiments, the catalyst bed consists of one or more segments of monolithic catalyst. In some embodiments, the catalyst bed contains two or more segments of monolithic catalyst. This includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 segments of monolithic catalyst. In some embodiments, the catalyst bed contains up to 100 segments of monolithic catalyst. In some embodiments, the catalyst bed contains from 2 to 100 segments of monolithic catalyst, including ranges therein.

Each of the one or more segments of monolithic catalyst may be about 7 centimeters (cm) to about 20 meters (m) in length. This includes ranges of about 7 cm to about 15 m, about 7 cm to about 10 m, about 7 cm to about 5 m, about 7 cm to about 1 m, about 7 cm to about 75 cm, about 7 cm to about 50 cm, about 7 cm to about 25 cm, about 10 cm to about 20 m, about 10 cm to about 15 m, about 10 cm to about 10 m, about 10 cm to about 5 m, about 10 cm to about 1 m, about 10 cm to about 75 cm, about 10 cm to about 50 cm, about 10 cm to about 25 cm, about 15 cm to about 20 m, about 15 cm to about 15 m, about 15 cm to about 10 m, about 15 cm to about 5 m, about 15 cm to about 1 m, about 15 cm to about 75 cm, about 15 cm to about 50 cm, or about 15 cm to about 25 cm. In some embodiments, each of one or more segments of monolithic catalyst is about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 cm in length, including increments therein. In some embodiments, each of one or more segments of monolithic catalyst is about 1, 2, 3, 4, 5, 6, 7, 8, 9, 10, 11, 12, 13, 14, 15, 16, 17, 18, 19, or 20 m in length, including increments therein.

The monolithic catalyst may contain a monolithic honeycomb structure containing a plurality of channels aligned side by side wherein each channel contains an inlet positioned at a first terminus of the channel, an outlet positioned at a second terminus of the channel, and openings positioned along the channel in the direction of fluid flow through the channel for transverse fluid flow in and/or out of the channel; and a layer of catalyst coating the honeycomb structure. In some embodiments, some of the openings are accompanied by a projection of channel wall toward the interior of the channel. In some embodiments, each of the openings is accompanied by a projection of channel wall toward the interior of the channel. In some embodiments, at least 1% of the openings are accompanied by a projection of channel wall toward the interior of the channel. This includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% of the openings are accompanied by a projection of channel wall toward the interior of the channel. In some embodiments, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% of the openings are accompanied by a projection of channel wall toward the interior of the channel. In some embodiments, 0 to 100% of the openings are accompanied by a projection of channel wall toward the interior of the channel.

Without being bound to any one particular theory, it is believed that the openings positioned along the channel in the direction of fluid flow and/or the projections of the channel wall create turbulent fluid flow through each channel and enable homogeneous distribution of mass and heat flow by fluid communication between adjacent channels within the monolithic catalyst. In some embodiments, the monolithic catalyst described herein is compatible for use with exothermic or endothermic chemical reactions. In some embodiments, uneven distribution of heat is minimized or avoided within the channels. In some embodiments, the temperature distribution within the bed is less than 50° C. In some embodiments, less than 10° C. can be observed for the catalyst beds described herein when in use, either longitudinally through the entire bed or radially from the edge of the bed to the center of the bed. In further embodiments, flow characteristics and honeycomb bed structures can be varied, as long as the resulting temperature distribution falls within the desirable range, such as, but not limited to, <10° C.

The monolithic catalyst may contain a layer of catalyst coating the interior of each channel. The layer of catalyst may contain a refractory metal oxide support impregnated with metal. The refractory metal oxide support may include a compound selected from alumina, silica, zirconia, titania, or a combination of any two or more thereof. In some embodiments, the layer of catalyst contains about 1 wt. % to about 50 wt. % metal. This includes ranges of about 1 wt. % to about 45 wt. % metal, about 1 wt. % to about 40 wt. % metal, about 1 wt. % to about 35 wt. % metal, about 1 wt. % to about 30 wt. % metal, about 5 wt. % to about 50 wt. % metal, about 5 wt. % to about 45 wt. % metal, about 5 wt. % to about 40 wt. % metal, about 5 wt. % to about 35 wt. % metal, about 5 wt. % to about 30 wt. % metal, about 10 wt. % to about 50 wt. % metal, about 10 wt. % to about 45 wt. % metal, about 10 wt. % to about 40 wt. % metal, about 10 wt. % to about 35 wt. % metal, or about 10 wt. % to about 30 wt. % metal. In some embodiments, the layer of catalyst contains about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt. % metal. In some embodiments, the layer of catalyst contains a refractory metal oxide support impregnated with a metal such as silver, copper, cobalt, nickel, or gold, or a combination of any two or more thereof. The metal may be in its elemental form, a salt form, or in the form of a metal oxide. In some embodiments, the layer of catalyst contains an alumina-based support impregnated with a metal such as silver, copper, cobalt, nickel, or gold, or a combination of any two or more thereof. In some embodiments, the layer of catalyst contains an alumina-based support impregnated with silver, copper, cobalt, nickel, or a combination of any two or more thereof. In some embodiments, the layer of catalyst contains an alumina-based support impregnated with silver. In some embodiments, the layer of catalyst contains an alumina-based support impregnated with copper. In some embodiments, the layer of catalyst contains an alumina-based support impregnated with cobalt. In some embodiments, the layer of catalyst contains an alumina-based support impregnated with nickel. In some embodiments, the layer of catalyst contains an alumina-based support impregnated with gold.

The thickness of the layer of catalyst on the honeycomb structure may be measured by units of weight of the component (e.g., the layer of catalyst, or in some embodiments, the refractory metal oxide support impregnated with metal) per unit of volume of the honeycomb and expressed as g/in³. The thickness of the layer of catalyst on the honeycomb structure may be from about 0.1 g/in³ to about 10 g/in³. This includes from about 0.1 g/in³ to about 8 g/in³, from about 0.1 g/in³ to about 5 g/in³, from about 0.1 g/in³ to about 3 g/in³, from about 0.1 g/in³ to about 1 g/in³, from about 0.1 g/in³ to about 0.8 g/in³, from about 0.1 g/in³ to about 0.5 g/in³, from about 0.5 g/in³ to about 10 g/in³, from about 0.5 g/in³ to about 8 g/in³, from about 0.5 g/in³ to about 5 g/in³, from about 0.5 g/in³ to about 3 g/in³, from about 0.5 g/in³ to about 1 g/in³, from about 1 g/in³ to about 10 g/in³, from about 1 g/in³ to about 5 g/in³, or from about 5 g/in³ to about 10 g/in³. In some embodiments, the thickness of the layer of catalyst on the honeycomb structure is about 0.1, 0.2, 0.3, 0.4, 0.5, 0.6, 0.7, 0.8, 0.9, 1.0, 1.3, 1.5, 1.8, 2.0, 2.3, 2.5, 2.8, 3.0, 3.3, 3.5, 3.8, 4.0, 4.3, 4.5, 4.8, 5.0, 5.3, 5.5, 5.8, 6.0, 6.3, 6.5, 6.8, 7.0, 7.3, 7.5, 7.8, 8.0, 8.3, 8.5, 8.8, 9.0, 9.3, 9.5, 9.8, or 10 g/in³, including increments therein.

The monolithic honeycomb structure may be a metallic monolith or a ceramic cordierite monolith. The monolithic honeycomb structure may contain cordierite, aluminum titanite, silicon carbide, aluminum carbide, or a combination of any two or more thereof. In some embodiments, the monolithic honeycomb structure contains cordierite, steel, or aluminum. In some embodiments, the monolithic honeycomb structure contains cordierite. In some embodiments, the monolithic honeycomb structure contains aluminum or steel. In some embodiments, the monolithic honeycomb structure contains aluminum titanite. In some embodiments, the monolithic honeycomb structure contains silicon carbide. In some embodiments, the monolithic honeycomb structure contains aluminum carbide. Examples of commercially available monolithic honeycomb structure include, but are not limited to, LS®-Design and PE™-Design catalyst supports from Emitec GmbH. The monolithic honeycomb structure may contain steel, carbon steel, stainless steel, copper, aluminum, tin, nickel, cobalt, magnesium, manganese, titanium, zirconium or tungsten, or any combination of two or more thereof.

The catalyst bed may be partitioned into one or more zones. In some embodiments, the catalyst bed is partitioned into two, three, four, five, six, seven, eight, nine, or ten zones. A zone may contain either a honeycomb structure or a structure of a conventional packed bed. Two or more zones may be arranged in an intermingled fashion or be sequentially positioned. One non-limiting arrangement is shown in Example 4.

The monolithic honeycomb structure may include a wall along the outer perimeter of the honeycomb structure. In some embodiments, the wall allows for the honeycomb structure to be affixed (e.g., welded) onto a reactor system (e.g., reactor tube). In some embodiments, the wall may be longer than the remainder of the honeycomb structure, thereby providing a gap devoid of catalyst between two sequentially positioned honeycomb structures.

Conventional catalyst beds may be contained within a thin bed tube (in some embodiments, about 1-3 inches in diameter and about 10 meters in length). Such a bed tube may incorporate a set of segments of monolithic catalyst described herein, wherein the set has the same diameter as the bed tube. A non-limiting example of such a configuration is shown in Example 4.

The catalyst bed may further contain catalyst pellets. In some embodiments, the catalyst pellets may contain the same metal as the metal catalyst of the monolithic catalyst. In some embodiments, the catalyst pellets may contain a different metal as the metal catalyst of the monolithic catalyst. Catalyst pellets are known to those skilled in the art, and can be readily purchased from commercial vendors or prepared by published protocols. A non-limiting example of catalyst pellets is described in U.S. Pat. No. 8,987,482, hereby incorporated by reference in its entirety. Catalyst pellets may have the geometry of an extrudate, such as, but not limited to, a hollow extrudate, a star, a sphere, a ring, or a cylinder.

In some embodiments, the catalyst pellets and the one or more segments of monolithic catalyst are located in separate zones of the catalyst bed. In some embodiments, a zone containing one or more segments of monolithic catalyst is positioned to encounter fluid flow before a zone containing catalyst pellets. In some embodiments, a zone containing one or more segments of monolithic catalyst is positioned to encounter fluid flow after a zone containing catalyst pellets. In some embodiments, the catalyst pellets and the one or more segments of monolithic catalyst are located in separate zones in an alternating pattern. In some embodiments, the catalyst bed contains two or more segments of monolithic catalyst and the catalyst bed further contains a gap devoid of catalyst positioned between each of the two or more segments of monolithic catalyst.

In another aspect, provided herein are methods to prepare the monolithic catalysts described herein. The method may include coating a monolithic honeycomb structure with a slurry of supported catalyst to form a coated honeycomb structure; and drying the coated honeycomb structure with heated forced air to produce the monolithic catalyst. In some embodiments, the coating is performed by dipping the monolithic honeycomb structure into the slurry of supported catalyst. In some embodiments, the coating is performed by applying a wash-coat of supported catalyst to the monolithic honeycomb structure. In some embodiments, the coating step forms a layer of supported catalyst on the interior of each channel.

The method may include coating a monolithic honeycomb structure with a layer of alumina-based support to form a pre-coated monolithic honeycomb structure; impregnating the layer of alumina-based support with a metal catalyst to form an impregnated monolithic honeycomb structure; and drying the impregnated monolithic honeycomb structure with heated forced air to produce the monolithic catalyst. In some embodiments, the coating step and impregnating step form a layer of alumina-based support impregnated with metal catalyst on the interior of each channel.

In another aspect, provided herein are methods of using the monolithic catalysts described herein and catalyst beds containing the same. The methods may include preparing ethylene oxide by contacting a feed gas containing ethylene with a monolithic catalyst described herein to form ethylene oxide. The methods may include performing direct epoxidation of ethylene by contacting a feed gas containing ethylene with a monolithic catalyst described herein to form ethylene oxide.

The methods may include partial oxidation of a feed gas. The methods may include dehydrogenation of a feed gas. Non-limiting examples include, but are not limited to, a butane to maleic anhydride process and a propionaldehyde oxidation to propionic acid process.

The methods may improve space-time-yield (STY) of ethylene oxide production, with the methods including contacting a feed gas containing ethylene with a catalyst bed described herein and forming ethylene oxide by direct epoxidation.

The methods may include the use of a promoter. Examples of promoters include, but are not limited to, rhenium, tungsten, lithium, cesium, sulfur, and any combination of two or more thereof. In some embodiments, the monolithic catalyst further contains a promoter. In some embodiments, the refractory metal oxide support is further impregnated with a promoter.

The present invention, thus generally described, will be understood more readily by reference to the following examples, which are provided by way of illustration and are not intended to be limiting of the present invention.

### EXAMPLES

### Example 1. Pressure Drop Measurements of Monolithic Structures Versus Pellets in a Packed Bed Geometry.

A pressure drop measurement apparatus was constructed using an optically transparent polycarbonate tube of 1.5 inches inside diameter with a single layer of woven steel mesh affixed to one end. This tube was mounted to a Superflow SF-1020 air flow bench filled with either monolith cores or ceramic pellets at specified bed heights. Pressure drop measurements were conducted at room temperature using air flow rates spanning 15 standard cubic feet per minute (SCFM) to 170 SCFM. Replicate experiments were conducted to ensure statistical significance.

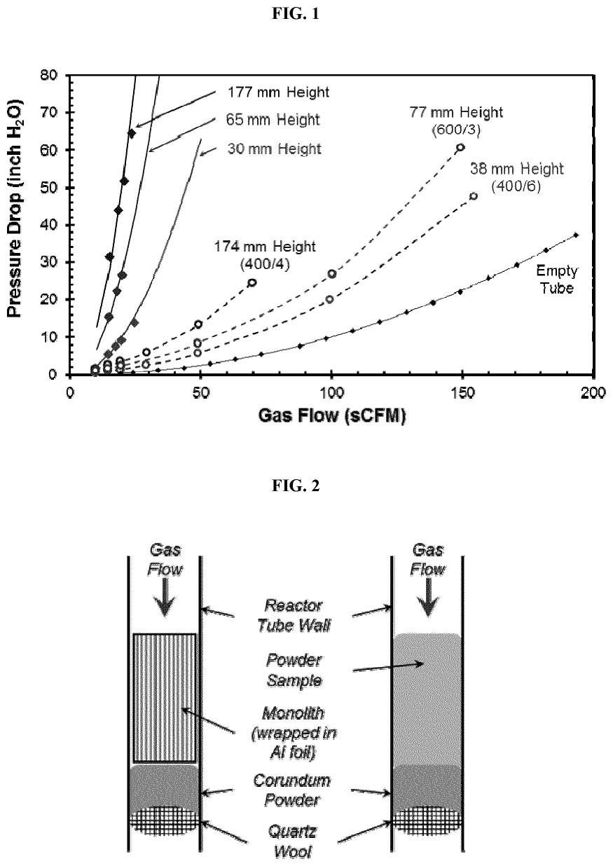

Uncoated ring-shaped ceramic pellets, typical of commercial ethylene epoxidation catalysts, were loaded into the tube at bed heights spanning 3.0 cm to 17.7 cm, and pressure drops were measured using the method described above, and the results are presented in FIG. 1 (the ceramic pellets are comparative and are represented as the solid lines/diamonds in FIG. 1).

Uncoated cordierite monolith sections of 1.5 inch outside diameter were cored from larger monolith bricks consisting of square, straight channels with channel densities of 400 cpsi and 600 cpsi (cells per square inch or channels per square inch), and wall thicknesses of 4 mil, 6 mil, and 3 mil. Cores were stacked in the tube at various heights spanning 3.8 cm to 29.0 cm, without spacers or alignment of channels. Pressure drops were measured using the method described above, and the results are presented in FIG. 1 (the monoliths are the dashed lines/circles in FIG. 1).

The data shown in FIG. 1 demonstrates the dramatic reduction in pressure drop that monolith geometries (dashed lines/circles) offered compared to similar bed heights of pellets (solid lines/diamonds) at identical flow conditions. As much as 10× higher pressure drop is demonstrated with pellet bed geometries (representative of a conventional production geometry) compared to the monolith geometry. A drop in pressure is desirable as it leads to decreased energy costs and allows the passage of more feed gas, thereby enhancing production rate.

### Example 2. Investigation of Monolithic Catalysts Versus Catalytic Pellets in a Packed Bed Geometry.

A silver complex solution was prepared according to U.S. Pat. No. 8,629,079: Col. 26, Example 1.2. Slurries were coated onto cordierite monolith cores by dipping into the slurry solution, drying with heated forced air, and weighing the dried cores to determine coating weight. Coating slurries were diluted with water to achieve lower coating weights when necessary. The cores were then heated to 280° C. to activate the silver complex as described in patent WO 2012/140614A1. Coating weights were from 2.0 g/in³ to 8.0 g/in³, with silver contents as high as 30% wt.

Testing was conducted in a high throughput experimentation unit which replicated direct ethylene epoxidation feed and reactor conditions, and measured downstream gas compositions via gas chromatography to determine selectivity and conversion. The unit had 48 separate reactor tubes arranged on a temperature controlled plate. During operation, reactive conditions were applied to each tube in sequentially for each process condition.

Each reactor tube was loaded as shown in FIG. 2. Quartz wool was loaded first, then covered with a guard bed of Corundum powder (125-160 micron particle size) to establish a consistent pressure drop across all reactor tubes. Powder or monolith cores were loaded on top of this guard bed in each of the reactor tubes. Such a Corundum guard bed may not be present in production reactor tubes, compared to the reactor tubes used for testing shown here.

Powder control samples were prepared as described in U.S. Pat. No. 8,629,079: Col. 26, Example 1. Pellets were then crushed and dry sieved to a (-) 45 mesh particle size. Powders were then pressed into a pellet and ground to a particle size of 500-1000 microns. Baseline pressure drop measurements on the guard bed demonstrated a pressured drop variability of less than 20% across a plate.

Monolith cores were prepared in lengths of 10 mm, 20 mm, and 40 mm. Each core was wrapped in aluminum foil to ensure a snug fit in the reactor tube, and loaded on top of the guard bed. Aluminum foil was also tested separately to confirm it did not impact the measurement.

Testing began after conditioning all loaded samples at 250° C., 1.0 bar-gauge, at 2000 Nm³/m³/hr GHSV (gas hourly space velocity) for 70 hrs using the reaction feed gas, and confirming steady state performance was achieved. The reaction feed gas included 35% ethylene, 7% oxygen, and other inactive gases (such as, but not limited to, nitrogen or methane). Conditioning and initial testing was conducted in the absence of a gas phase moderator to avoid the risk of premature poisoning of monolith samples.

The tests were conducted at 200° C., 220° C., and 240° C. GHSV was varied by changing flow rate, and by using replicate samples with different bed heights. Tests were conducted at GHSV values in the spanning 1000-10000 Nm³/m³/hr (Nm³=normal cubic meters, which is the volume of an ideal gas at normal conditions (1 bar and 273.15 K)).

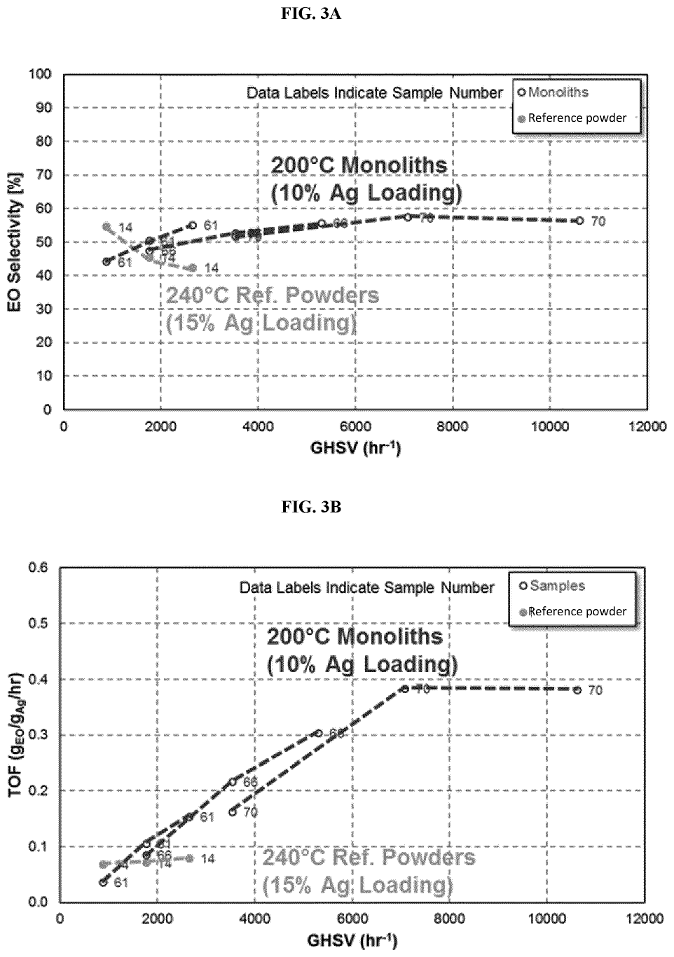

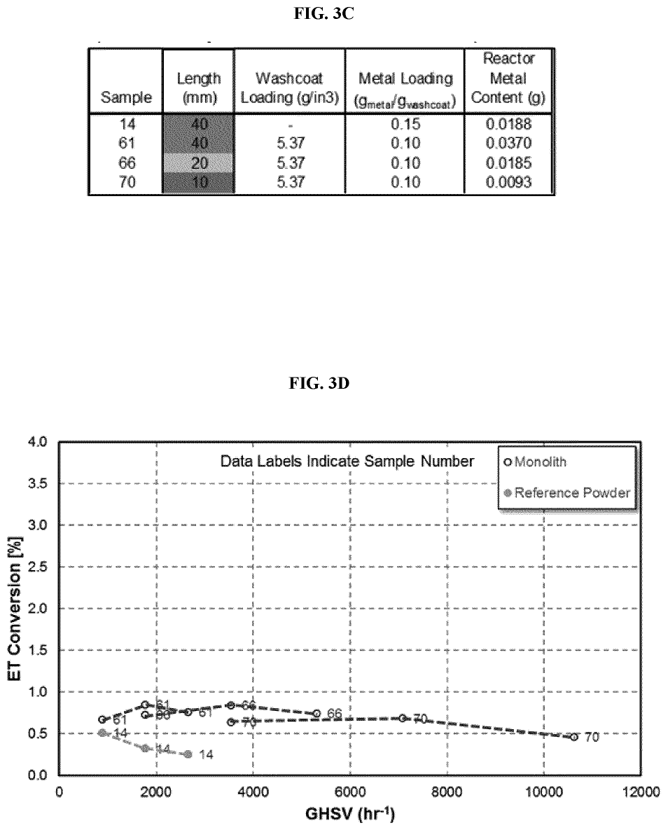

The results comparing monolith and powder bed geometries with identical catalyst chemistries and test conditions are shown in FIGS. 3A-3D. In the absence of a gas phase moderator, monolith geometry catalysts provide a substantial increase in selectivity, turnover frequency, and conversion compared to powder beds, while operating at lower optimum temperature and with less silver content. Monolith samples were non-selective at 240° C., and powders were inactive at 200° C. Reynold's number calculations indicate laminar flow for both sample geometries across the entire flow rate range that was investigated. Ethylene oxide selectivity values are considerably lower than production values due to the absence of gas phase moderator dosing.

### Example 3. Use Without A Heat Exchanger

The monolith structure operates non-adiabatically, thus eliminating the need for a dedicated heat exchanger. This is different from US2011060149 which requires an external heat removal device. Non-adiabatic operation is accomplished by the use of turbulence generating metallic foil monoliths, which allow for (a) turbulence generating channel structures to promote convective heat transfer via turbulent flow, and (b) high channel densities approaching 1000 cpsi to promote conductive heat transfer.

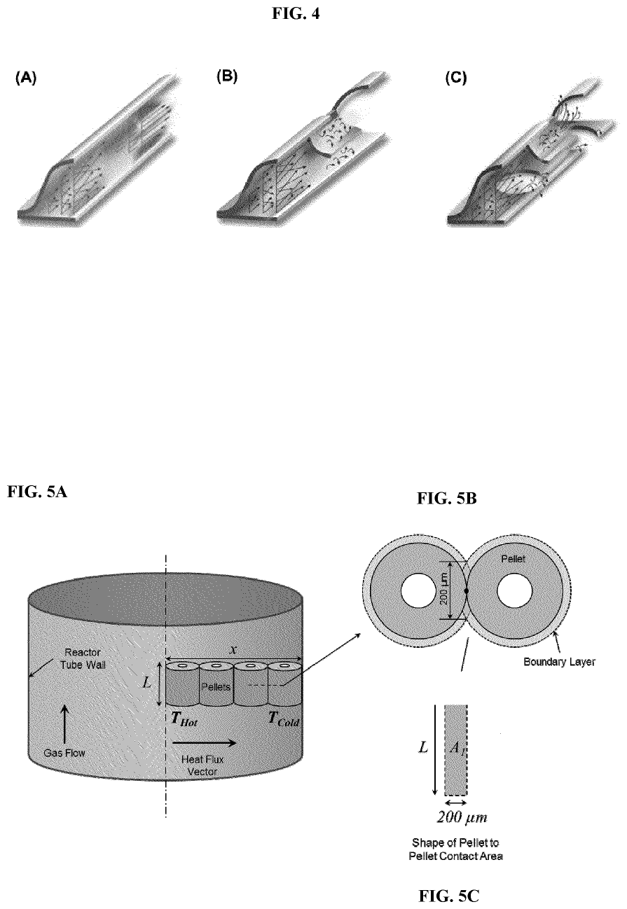

Industrial scale packed bed geometry direct ethylene epoxidation reactors operate with a Reynolds number of approximately 29,500 representing strongly turbulent flow. In these designs, the majority of the exothermic heat load is removed via convection currents in the gas phase. For monolith-based designs, a variety of turbulence generating channel geometries are commercially available and can be used in this application. In this example, we consider three available geometries as shown in FIG. 4. The SC geometry represents a baseline straight channel geometry and is not considered “turbulence generating”. The LS geometry introduces gaps and accompanying protruding blades of approximately 5 mm in length periodically spaced approximately every 6-8 mm along the channel axis to disrupt gas flow. The LS/PE geometry (combination of LS and PE geometries) employs identical gaps and blades, but also introduces holes to allow gas transport between channels, which allows for radial gas transport throughout the monolith brick.

The blades in the LS and LS/PE structures impede the formation of fully developed flow by introducing flow disruption along a distance described by the entrance length, according to the formula:

L=0.06*Re*Dₕ

In the above formula, Rₑ is Reynolds's number; Dₕ is hydraulic diameter. Within the entrance length regions, flow is considered turbulent. Table 1 shows the entrance length needed before a fully developed laminar flow pattern is established, for the straight channel (SC) geometry and the LS structure geometry.

As shown in Table 1, the entrance length for the SC geometry is short enough that fully developed laminar flow will occur within a few centimeters of entering the monolith. The LS geometry, however, does not achieve fully developed flow at any location, because the entrance is substantially longer than the periodic spacing of flow disrupting blades (6-8 mm).

The LS/PE structure exhibits quantitatively similar turbulence due to identical blade spacing and hydraulic diameters, but will offer further enhanced convective heat transfer due to radial transport of gas as described above with regard to the monolith channel structures of FIG. 4.

TABLE 1
Results of flow calculations for metal monolith channel geometries
shown in FIG. 4, subject to industrial reactor conditions

| | | |
|---|---|---|
| Channel | Reynold's | Entrance Length |
| Density | Number | (mm) |

| (cpsi) | SC | LS | SC | LS |
|---|---|---|---|---|
| 200 | 1662 | 1000 | 153.0 | 55.3 |
| 300 | 1383 | 828 | 104.6 | 37.5 |
| 400 | 1206 | 724 | 78.6 | 28.3 |
| 500 | 1095 | — | 64.2 | — |
| 600 | 1008 | — | 53.9 | — |
| 800 | 886 | — | 40.9 | — |
| 900 | 840 | — | 36.6 | — |
| 1000 | 802 | — | 33.1 | — |

The calculations indicate that turbulent flow conditions identical to those of pellet beds can also be achieved in monolith structures. Since the majority of the exothermic heat load is relieved through gas phase convection, similar thermal performance can be expected from turbulence generating monolith designs that are sized appropriately to the reactor conditions.

The thermal conduction through a solid monolith structure is also more efficient than radial thermal conduction through an industrial pellet bed, due to the superior heat transfer characteristics of metallic foils compared to ceramic pellets. Although this represents a minor contribution to heat removal under turbulent flow conditions, it is a significant contributor to heat removal under the less desirable laminar flow regime, and is considered here for completion.

The solid phase thermal conductivity of pellet and monolith reactor geometries is compared by applying the one-dimensional version of Fourier's Law of heat conduction, given by:

$$\dot{Q} = \frac{{T}_{1} - {T}_{2}}{( \frac{Δ x}{k A} )} = \frac{{T}_{1} - {T}_{2}}{R}$$

In the above formula, T₁ and T₂ are hot and cold temperatures, respectively, Δx is the distance over which heat transport is measured, k is the thermal conductivity of the solid medium, A is the heat flux cross sectional area, and R is the thermal resistance of the solid medium. Thermal resistance is inversely related to thermal conductivity, and represents the ability of a material to resist heat flow.

For simplicity, this analysis assumes negligible thermal resistance between contacting surfaces of pellets (i.e., perfect thermal contact between pellets). This assumption represents the maximum theoretical limit for thermal conductivity between solid pellets, and substantially lower thermal conductivity between pellets is expected in practice. Negligible thermal resistance between contacting monolith channels is also assumed (i.e., perfect thermal contact between channel walls). This assumption is reasonable in practice, because monoliths are assembled by welding foil walls together to increase rigidity and thermal conductivity. It is further assumed that both monolith bricks and ceramic pellets have perfect thermal contact to the reactor wall.

The first pellet geometry considered is shown in FIG. 5, and represents cylindrical ceramic pellets oriented side by side with long axes parallel to the direction of gas flow and perpendicular to the direction of thermal conduction. A hot spot is located along the center axis of the reactor with cooling at the reactor wall. This geometry is referred to as A₁. A boundary layer is assumed to exist around the pellets to account for surface roughness and shape irregularities that define a nonzero rectangular contact area approximately 200 μm in width and spanning the full length of a pellet.

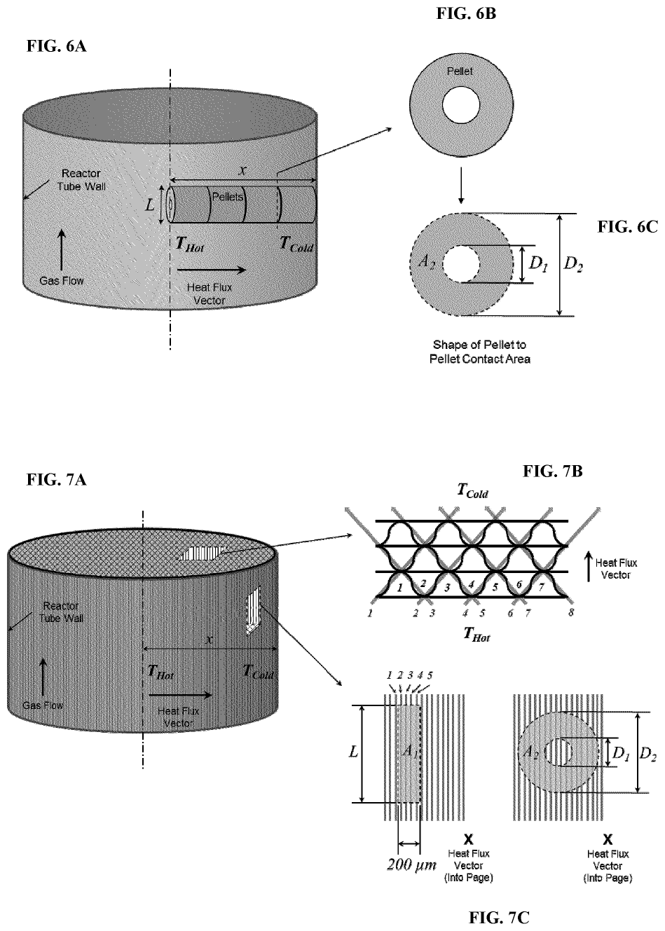

The second pellet geometry considered is shown in FIG. 6, and represents cylindrical ceramic pellets oriented end to end with long axis perpendicular to the direction of gas flow and parallel to the direction of thermal conduction. A hot spot is located along the center axis of the reactor with cooling at the reactor wall. This geometry is referred to as A₂. The contact area between pellets in the A₂ geometry is an annulus defined by the inside and outside diameters of a ceramic pellet.

The third geometry considered is shown in FIG. 7, and consists of a straight channel monolith brick oriented with the channel axes parallel to the direction of gas flow. The assumed channel density is 1000 cpsi and the assumed channel wall thickness is 50.8 μm (0.002 inch). In this geometry, radial heat transfer occurs along channel walls.

To compare directly with the pellet geometries, the cross sectional contact areas of the A₁ and A₂ geometries are overlaid on the foil walls of the monolith in the radial direction to determine the volume of metal foil participating in heat conduction in those cross sectional areas. These monolith equivalent contact area geometries are referred to as A_{l mono} and A_{2 mono}, respectively.

The cumulative cross sectional area of monolith walls participating in heat conduction through a cross sectional area equivalent to area Aₗ is provided by:

$${A}_{1 m o n o} = ( \frac{{A}_{1}}{c} + 1 ) L d$$

where c is the channel density (1.55 channels/mm²), d is the foil wall thickness, and L is the pellet height. The cumulative cross sectional area of monolith walls participating in heat conduction through a cross sectional area equivalent to area A₂ is provided by:

$${A}_{2 m o n o} = 0.25 π ( \frac{{A}_{2}}{c} + 1 ) {D}_{2} d$$

where D₂ is the outside diameter of a pellet.

Using areas Aₗ, A_{1 mono}, A₂, and A_{2 mono}, the thermal resistances can be calculated for comparison according to:

$${R}_{i} = \frac{x}{{k}_{i} {A}_{i}}$$

The thermal conductivity (10 of an a-alumina pellet and an aluminum monolith wall are assumed to be 35 W/m²-K and 205 W/m²-K, respectively. The calculated thermal resistances for an arbitrary heat transfer distance (x) of 10 mm are shown in Table 2.

The heat transfer resistances indicate that metallic monoliths have at least 50% better thermal conductivity compared to industrial ceramic pellet beds, despite less contact area and calculation assumptions that inflate pellet bed thermal conduction values.

TABLE 2
Calculated Thermal Resistances for Pellet Bed and Monolith Bed

| | Heat Flux | Thermal |
|---|---|---|
| Geometry | Cross Section (mm²) | Resistance (K/W) |
| A₁ | 1.59 | 1.80 × 10⁻¹ |
| A_{1 mono} | 0.807 | 6.06 × 10⁻² |
| A₂ | 51.4 | 5.55 × 10⁻³ |
| A_{2 mono} | 11.4 | 4.29 × 10⁻³ |

### Example 4. Hybrid Reactor Geometry.

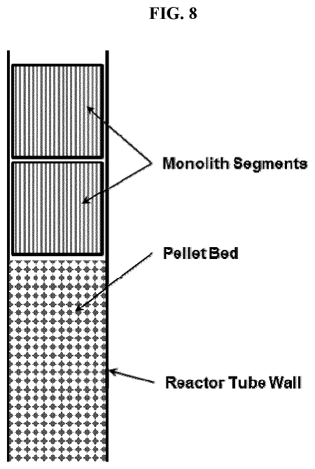

A tubular metal reactor was partially filled with conventional packed bed catalyst pellets, with the remainder of the reactor volume filled with metallic monolith segments coated with catalytically active washcoat prepared as described in Example 2. This reactor configuration (FIG. 8) achieved some benefits of a monolith geometry with the lower risks of a conventional packed bed geometry, and may offer a better drop-in solution for existing plants with downstream constraints on space velocity or mass flow. This reactor configuration achieved some benefits of both a monolith geometry (e.g., lower optimum temperature, lower pressure drop, higher turnover frequency, lower Ag content, potential for alternative catalyst/support chemistries, etc.) and a packed pellet bed geometry (e.g., tolerance to combustion moderator dosing, wider operating temperature).

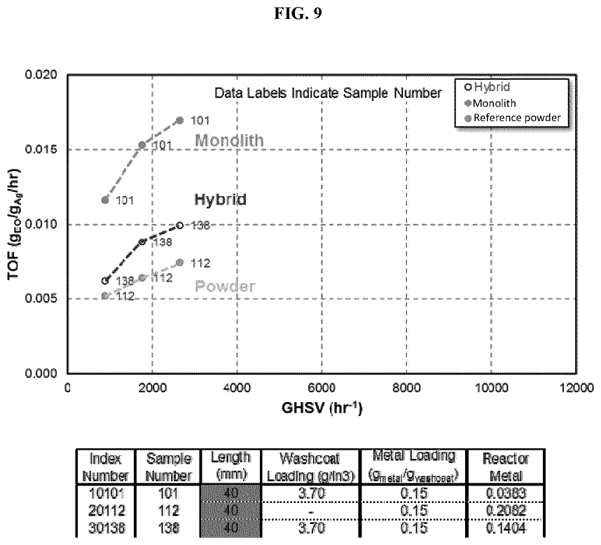

Using the same testing procedures described in Example 2, hybrid reactor configurations were tested alongside powder bed and monolith bed geometries, utilizing identical catalyst chemistries. Results are shown in FIG. 9. The hybrid sample data corresponded to a reactor tube loaded halfway with crushed production catalyst pellets, and halfway with a coated monolith.

The hybrid reactor geometry exhibited performance between that of a pure monolith and a pure powder bed geometry.

Para. A. A catalyst bed comprising one or more segments of monolithic catalyst comprising:
a monolithic honeycomb structure comprising a plurality of channels aligned side by side wherein each channel comprises an inlet positioned at a first terminus of the channel, an outlet positioned at a second terminus of the channel, and openings positioned along the channel in the direction of fluid flow through the channel for transverse fluid flow in and/or out of the channel; and
a layer of catalyst coating the honeycomb structure.

Para. B. The catalyst bed of Para. A, wherein each of the openings is accompanied by a projection of channel wall toward the interior of the channel.

Para. C. The catalyst bed of Para. A or Para. B, wherein fluid flow is turbulent through each channel.

Para. D. The catalyst bed of any one of Paras. A-C, wherein accumulation of heat is minimized or avoided within the channels.

Para. E. The catalyst bed of any one of Paras. A-D, wherein the layer of catalyst coats the interior of each channel.

Para. F. The catalyst bed of any one of Paras. A-E, wherein the layer of catalyst comprises a refractory metal oxide support impregnated with metal.

Para. G. The catalyst bed of Para. F, wherein the refractory metal oxide support comprises a compound selected from alumina, silica, zirconia, titania, or a combination of any two or more thereof.

Para. H. The catalyst bed of Para. F or Para. G, wherein the layer of catalyst comprises about 1 wt. % to about 50 wt. % metal.

Para. I. The catalyst bed of any one of Paras. F-H, wherein the layer of catalyst comprises about 10 wt. % to about 30 wt. % metal.

Para. J. The catalyst bed of any one of Paras. F-I, wherein the layer of catalyst comprises an alumina-based support impregnated with a metal selected from silver, copper, cobalt, nickel, or gold, or a combination of any two or more thereof.

Para. K. The catalyst bed of any one of Paras. A-J, wherein the monolithic honeycomb structure comprises cordierite, steel, or aluminum.

Para. L. The catalyst bed of any one of Paras. A-K, wherein each of the one or more segments is about 7 centimeters to about 20 meters in length.

Para. M. The catalyst bed of any one of Paras. A-L, wherein each of the one or more segments is about 15 centimeters to about 25 centimeters in length.

Para. N. The catalyst bed of any one of Paras. A-M, wherein the catalyst bed is partitioned into one or more zones.

Para. O. The catalyst bed of any one of Paras. A-N, wherein the catalyst bed further comprises catalyst pellets.

Para. P. The catalyst bed of Para. 0, wherein the catalyst pellets and the one or more segments of monolithic catalyst are located in separate zones.

Para. Q. The catalyst bed of Para. P, wherein a zone comprising one or more segments of monolithic catalyst is positioned to encounter fluid flow before a zone comprising catalyst pellets.

Para. R. The catalyst bed of Para. P, wherein the catalyst pellets and the one or more segments of monolithic catalyst are located in separate zones in an alternating pattern.

Para. S. The catalyst bed of any one of Paras. A-R, wherein the catalyst bed comprises two or more segments of monolithic catalyst, and the catalyst bed further comprises a gap devoid of catalyst positioned between each of the two or more segments of monolithic catalyst.

Para. T. The catalyst bed of any one of Paras. A-S for the preparation of ethylene oxide.

Para. U. A monolithic catalyst comprising:
a monolithic honeycomb structure comprising a plurality of channels aligned side by side; and each channel comprises an inlet positioned at a first terminus of the channel, an outlet positioned at a second terminus of the channel, and openings positioned along the channel in the direction of fluid flow through the channel for transverse fluid flow in and/or out of the channel; and
a layer of catalyst coating the honeycomb structure.

Para. V. The monolithic catalyst of Para. U, wherein each of the openings is accompanied by a projection of channel wall toward the interior of the channel.

Para. W. The monolithic catalyst of Para. U or Para. V, wherein fluid flow is turbulent through each channel.

Para. X. The monolithic catalyst of any one of Paras. U-W, wherein accumulation of heat is minimized or avoided within the channels.

Para. Y. The monolithic catalyst of any one of Paras. U-X, wherein the layer of catalyst coats the interior of each channel.

Para. Z. The monolithic catalyst of any one of Paras. U-Y, wherein the layer of catalyst comprises a refractory metal oxide support impregnated with metal.

Para. AA. The monolithic catalyst of Para. Z, wherein the refractory metal oxide support comprises a compound selected from alumina, silica, zirconia, titania, or a combination of any two or more thereof.

Para. AB. The monolithic catalyst of Para. Z or Para. AA, wherein the layer of catalyst comprises about 1 wt. % to about 50 wt. % metal.

Para. AC. The monolithic catalyst of any one of Paras. Z-AB, wherein the layer of catalyst comprises about 10 wt. % to about 30 wt. % metal.

Para. AD. The monolithic catalyst of any one of Paras. Z-AC, wherein the layer of catalyst comprises an alumina-based support impregnated with a metal selected from silver, copper, cobalt, nickel or gold, or a combination of two or more thereof.

Para. AE. The monolithic catalyst of any one of Paras. Z-AD, wherein the monolithic honeycomb structure comprises cordierite, steel, or aluminum.

Para. AF. A method to prepare ethylene oxide, the method comprising:
contacting a feed gas comprising ethylene with a monolithic catalyst to form ethylene oxide;
wherein:
   the monolithic catalyst comprises:
      a monolithic honeycomb structure comprising a plurality of channels, each channel comprising openings positioned along the channel in the direction of fluid flow through the channel; and
      a layer of catalyst coating the monolithic honeycomb structure.

Para. AG. The method of Para. AF, wherein each of the openings is accompanied by a projection of channel wall toward the interior of the channel.

Para. AH. The method of Para. AF or Para. AG, wherein fluid flow is turbulent through each channel.

Para. AI. The method of any one of Paras. AF-AH, wherein accumulation of heat is minimized or avoided within the channels.

Para. AJ. The method of any one of Paras. AF-AI, wherein the layer of catalyst coats the interior of each channel.

Para. AK. The method of any one of Paras. AF-AJ, wherein the layer of catalyst comprises a refractory metal oxide support impregnated with metal.

Para. AL. The method of Para. AK, wherein the refractory metal oxide support comprises a compound selected from alumina, silica, zirconia, titania, or a combination of any two or more thereof.

Para. AM. The method of Para. AK or Para. AL, wherein the layer of catalyst comprises about 1 wt. % to about 50 wt. % metal.

Para. AN. The method of any one of Paras. AK-AM, wherein the layer of catalyst comprises about 10 wt. % to about 30 wt. % metal.

Para. AO. The method of any one of Paras. AK-AN, wherein the layer of catalyst comprises an alumina-based support impregnated with a metal selected from silver, copper, cobalt, nickel or gold, or a combination of two or more thereof.

Para. AP. The method of any one of Paras. AF-AO, wherein the monolithic honeycomb structure comprises cordierite, steel, or aluminum.

Para. AQ. A method to prepare a monolithic catalyst, the method comprising:
coating a monolithic honeycomb structure with a slurry of supported catalyst to form a coated honeycomb structure; and
drying the coated honeycomb structure with heated forced air to produce the monolithic catalyst;
wherein:
   the monolithic honeycomb structure comprises a plurality of channels, each channel comprising openings positioned along the channel in the direction of fluid flow through the channel; and
   the supported catalyst comprises a refractory metal oxide support impregnated with metal.

Para. AR. The method of Para. AQ, wherein the coating is performed by dipping the monolithic honeycomb structure into the slurry of supported catalyst.

Para. AS. The method of Para. AQ, wherein the coating is performed by applying a wash-coat of supported catalyst to the monolithic honeycomb structure.

Para. AT. The method of any one of Paras. AQ-AS, wherein the coating step forms a layer of supported catalyst on the interior of each channel.

Para. AU. A method to prepare a monolithic catalyst, the method comprising:

coating a monolithic honeycomb structure with a layer of alumina-based support to form a pre-coated monolithic honeycomb structure;
impregnating the layer of alumina-based support with a metal catalyst to form an impregnated monolithic honeycomb structure; and
drying the impregnated monolithic honeycomb structure with heated forced air to produce the monolithic catalyst;
wherein the monolithic honeycomb structure comprises a plurality of channels, each channel comprising openings positioned along the channel in the direction of fluid flow through the channel.

Para. AV. The method of Para. AU, wherein the coating step and impregnating step form a layer of alumina-based support impregnated with metal catalyst on the interior of each channel.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A catalyst bed comprising one or more segments of monolithic catalyst comprising:
a monolithic honeycomb structure comprising a plurality of channels aligned side by side wherein each channel comprises an inlet positioned at a first terminus of the channel, an outlet positioned at a second terminus of the channel, and openings positioned along the channel in the direction of fluid flow through the channel for transverse fluid flow in and/or out of the channel; and
a layer of catalyst coating the honeycomb structure.

2. The catalyst bed of claim 1, wherein each of the openings is accompanied by a projection of channel wall toward the interior of the channel.

3. The catalyst bed of claim 1, wherein fluid flow is turbulent through each channel.

4. The catalyst bed of claim 1, wherein accumulation of heat is minimized or avoided within the channels.

5. The catalyst bed of claim 1, wherein the layer of catalyst coats the interior of each channel.

6. The catalyst bed of claim 1, wherein the layer of catalyst comprises a refractory metal oxide support impregnated with metal.

7. The catalyst bed of claim 6, wherein the refractory metal oxide support comprises a compound selected from alumina, silica, zirconia, titania, or a combination of any two or more thereof.

8. The catalyst bed of claim 6, wherein the layer of catalyst comprises about 1 wt. % to about 50 wt. % metal.

9. (canceled)

10. The catalyst bed of claim 6, wherein the layer of catalyst comprises an alumina-based support impregnated with a metal selected from silver, copper, cobalt, nickel, or gold, or a combination of any two or more thereof.

11. The catalyst bed of claim 1, wherein the monolithic honeycomb structure comprises cordierite, steel, or aluminum.

12. The catalyst bed of claim 1, wherein each of the one or more segments is about 7 centimeters to about 20 meters in length.

13. (canceled)

14. The catalyst bed of claim 1, wherein the catalyst bed is partitioned into one or more zones.

15. The catalyst bed of claim 1, wherein the catalyst bed further comprises catalyst pellets.

16. The catalyst bed of claim 15, wherein the catalyst pellets and the one or more segments of monolithic catalyst are located in separate zones.

17. The catalyst bed of claim 16, wherein a zone comprising one or more segments of monolithic catalyst is positioned to encounter fluid flow before a zone comprising catalyst pellets.

18. The catalyst bed of claim 16, wherein the catalyst pellets and the one or more segments of monolithic catalyst are located in separate zones in an alternating pattern.

19. The catalyst bed of claim 1, wherein the catalyst bed comprises two or more segments of monolithic catalyst, and the catalyst bed further comprises a gap devoid of catalyst positioned between each of the two or more segments of monolithic catalyst.

20. (canceled)

21. A monolithic catalyst comprising:
a monolithic honeycomb structure comprising a plurality of channels aligned side by side; and each channel comprises an inlet positioned at a first terminus of the channel, an outlet positioned at a second terminus of the channel, and openings positioned along the channel in the direction of fluid flow through the channel for transverse fluid flow in and/or out of the channel; and
a layer of catalyst coating the honeycomb structure.

22.-31. (canceled)

32. A method to prepare ethylene oxide, the method comprising:
contacting a feed gas comprising ethylene with a monolithic catalyst to form ethylene oxide;
wherein:
the monolithic catalyst comprises:
a monolithic honeycomb structure comprising a plurality of channels, each channel comprising openings positioned along the channel in the direction of fluid flow through the channel; and
a layer of catalyst coating the monolithic honeycomb structure.

33.-42. (canceled)

43. A method to prepare a monolithic catalyst of claim 21, the method comprising:
coating a monolithic honeycomb structure with a slurry of supported catalyst to form a coated honeycomb structure; and
drying the coated honeycomb structure with heated forced air to produce the monolithic catalyst;
wherein:
the monolithic honeycomb structure comprises a plurality of channels, each channel comprising openings positioned along the channel in the direction of fluid flow through the channel; and
the supported catalyst comprises a refractory metal oxide support impregnated with metal.

44-48. (canceled)
